(19) United States
(12) Patent Application Publication
(10) Pub. No.: US 2005/0233442 A1
(43) Pub. Date: Oct. 20, 2005

(54) Carrier for cell culture

(75) Inventors: Satoru Toda, Kanagawa (JP); Makoto Kato, Kanagawa (JP)

Correspondence Address: SUGHRUE MION, PLLC, 2100 PENNSYLVANIA AVENUE, N.W., SUITE 800, WASHINGTON, DC, 20037, US

(73) Assignee: FUJI PHOTO FILM CO., LTD.

(21) Appl. No.: 11/099,444

(22) Filed: Apr. 6, 2005

(30) Foreign Application Priority Data
Apr. 8, 2004 (JP) 2004-113977

(51) Int. Cl. C12N 5/02

(52) U.S. Cl. 435/325; 435/404

(57) ABSTRACT

A carrier for cell culture having multi-layer structure comprising a water-containing polymer gel layer, wherein the water-containing polymer gel layer has a thickness of 0.01 μm or more and 5 μm or less is provided. The carrier for cell culture preferably comprises a gel layer comprising an anionic polysaccharide and a polyvalent metal ion adjacent to the water-containing polymer gel and/or the most outer surface at the side for the cell culture of the carrier for cell culture is a cell-adhesive gel layer. A physical reinforcing means may be provided on one side of the water-containing polymer gel and/or a part unmodified with the cell-adhesive gel layer is provided on the most outer surface at the side for the cell culture. The carrier for cell culture has transparency and the cultured cell layer can be delaminated in a short time from the carrier.

### TECHNICAL FIELD

The present invention relates to a cell culture technology, specifically, a carrier for cell culture, a cell culture method using said carrier for cell culture, a cell culture obtained by said method, a cell layer lamination method using said cell culture, and laminated cell layers obtained by said cell layer lamination method.

### BACKGROUND ART

A water-containing polymer gel has a structure similar to that of a living body, and has a property of expanding or shrinking depending on external conditions such as temperature, acidity, and alkalinity. Accordingly, applications in the medical field, including a use as an artificial organ or tissue such as an artificial muscle or encapsulation of a drug therein to control an amount to be released, have been attempted, as well as applications as an anchorage of cell growth in a cell culture as a gel containing various kinds of cytokines and the like.

Poly-N-isopropylacrylamide (hereinafter referred to as PNIPAM in the specification), a temperature-sensitive polymer among a class of water-containing polymers, expands to exist as a liquid at low temperatures, whereas shrinks rapidly for gelation as a result of a phase transition at around 34° C. For conducting lamination of cultured cells, a method has been used so far wherein a cell cultured on gelated PNIPAM is laminated on another cell layer together with PNIPAM under a condition at 37° C., then the PNIPAM is removed by lowering the temperature under 34° C. for PNIPAM liquidation, thereby layering the cells directly to each other (Japanese Patent Publication (Kokoku) (Hei) No. 6-104061 (1994); Tatsuya Shimizu, Mitsuo Okano, Bioscience and Bioindustry 58(12), p 851(2000); Masayuki Yamato, Mitsuo Okano, Ringai, 56(1), p. 53 (2001); Masayuki Yamato, Mitsuo Okano, Materials Integration, 13(2), p 58 (2000); Masayuki Yamato, Ai Kushida, Mitsuo Okano, Tanpaku-shitu Kaku-san Koso [Protein, Nucleic Acid, Enzyme] 45(10), p. 72 (2000); Masayuki Yamato, Yukihiro Hirose, Masami Hashimoto, Mitsuo Okano, Tanpaku-shitu Kaku-san Koso [Protein, Nucleic Acid, Enzyme] 45(13), p. 162 (2000)).

When a cell culture is conducted on PNIPAM, cells generally proliferate as a monolayer, wherein extracellular matrix (hereinafter referred to as “ECM” in the specification) such as collagen are formed between the cells adjacent to each other. That is, adhesion to ECM is needed for the cell proliferation. However, the cultured cells have no adhesion to other cells on the upper side and between the cultured cells and PNIPAM as a base layer, where no ECM needed for cell adhesion is formed.

Accordingly, when monolayers of cell cultured on PNIPAM are laminated and the NIPAM is removed by liquidation under a condition of 34° C. or less to laminate the layers to achieve a direct adhesion of the cells to each other, cells laminated on upper side have insufficient anchorage for proliferation. Therefore, stable cell proliferation can not be expected. Further, liquidated PNIPAM, which act as a cytotoxic substance, is also sometimes observed to inhibit normal cell proliferation. Accordingly, the aforementioned technique is extremely unsuitable and unstable as a means for cell lamination.

In order to solve the above problems, researches have been conducted for establishment of cell culture using a medium wherein extracellular matrix component are layered on various gels and then gelled; and a cell culture system using a medium from which the waste gel can be easily removed after lamination. A carrier for a cell culture is proposed in Japanese Patent No. 3261456, which is characterized to contain a porous membrane wherein an alginic acid gel layer and extracellular matrix component gel layer or extracellular matrix component sponge layer are formed on the porous membrane. However, this carrier for a cell culture has a thick extracellular matrix layer which makes it impossible to store the carrier as a dried membrane. The thickness is also a burden for a cell lamination procedure to arise a problem that laminated cells are not stably obtained. Further, through the porous membrane disclosed in the publication, which has no transparency, growth state of living cells can not directly be observed visually, microscopically or the like during cell culture. Therefore the state of cells on the carrier for a cell culture is analogized to be in an equivalent state to the growth state of cells on a petri dish surrounding the carrier for a cell culture that is placed on the petri dish. However, the growth state of the cells surrounding the carrier for a cell culture on a petri dish does not always correspond to the state of the cells on the carrier, which arises a problem of insufficiency of cell proliferation or overconfluence. Therefore, a carrier for a cell culture which has such transparency as that of petri dish used for cell culture has been desired to accurately monitor the growth state of cells.

In addition, according to the method disclosed in the above publication, a cell culture is delaminated from the carrier for a cell culture by using EDTA aqueous solution for solubilization of the alginic acid gel layer, which arise another problem that cells are damaged through invasion by the EDTA aqueous solution. Therefore, a carrier for cell culture has been desired, which can reduce the damage to cells as low as possible when the cultured cells are delaminated from the carrier.

As a carrier for cell culture which comprises alginic acid or chitosan, a cell proliferation substrate comprising a polysaccharide and a cell adhesive protein is disclosed in Japanese Patent Unexamined Publication (KOKAI) No. 2002-536974. However, even if said cell proliferation substrate is used, an assured monitoring of the growth state of cells is impossible and delamination of a cultured cell layer is difficult.

### DISCLOSURE OF THE INVENTION

An object of the present invention is to provide a means for stable and easy cell lamination, and a means that can sufficiently make adhesion between cells in upper and lower layers when cell layers are laminated, which successfully paves the way to a cell lamination technique which has been considered unachievable under a condition in vitro except for some tissues such as skin.

Specifically, the object of the present invention is to provide a carrier for cell culture with transparency through which state of cultured cells can be directly observed, and from which a cultured cell layer can be delaminated in a short time.

The inventors of the present invention conducted various studies to achieve the foregoing objects. As a result, they found that a carrier for cell culture comprising a water-containing polymer gel layer having a specific thickness has transparency, thereby enables a direct observation of the state of cultured cells, and a short-time delamination of a cultured cell layer. They further found that operability at the cell layer delamination is improved by providing a physical reinforcing means at one side of the water-containing polymer gel layer and by other means. The present invention was achieved on the basis of these findings.

The present invention thus provides a carrier for cell culture having a multi-layer structure comprising a water-containing polymer gel layer, wherein the water-containing polymer gel layer has a thickness of 0.01 μm or more and 5 μm or less. According to preferred embodiments of the invention, there are provided the aforementioned carrier for cell culture, wherein the water-containing polymer gel in a liquid state has a viscosity of 1,000 mPa•s or more and 50,000 mPa•s or less; and the aforementioned carrier for cell culture, wherein the water-containing polymer gel is chitosan.

As further preferred embodiments of the present invention, there are provided: any one of the aforementioned carriers for cell culture which further comprises a gel layer comprising an anionic polysaccharide and a polyvalent metal ion, wherein said gel layer is adjacent to the water-containing polymer gel layer; the aforementioned carrier for cell culture, wherein the gel layer comprising an anionic polysaccharide and a polyvalent metal ion is an alginic acid gel; the aforementioned carrier for cell culture, wherein the gel layer comprising an anionic polysaccharide and a polyvalent metal ion is calcium alginate gel layer; any one of the aforementioned carriers for cell culture, wherein the most outer surface at the side for the cell culture is a cell-adhesive gel layer; the aforementioned carrier for cell culture, wherein the cell-adhesive gel is a gel comprising gelatin and/or collagen; and the aforementioned carrier for cell culture, wherein the cell-adhesive gel is a cell-adhesive collagen.

As still further preferred embodiments of the present invention, there are provided any one of the aforementioned carriers for cell culture, wherein a physical reinforcing means is provided on one of the surfaces of the water-containing polymer gel layer; any one of the aforementioned carriers for cell culture wherein the most outer surface at the side for the cell culture is the cell-adhesive gel layer, wherein an part that is unmodified with the cell-adhesive gel layer is provided on the most outer surface at the side for the cell culture; and the aforementioned carrier for cell culture which is sterilized by irradiation with one or more of electron beam, γ-ray, and ultraviolet ray.

From other aspects, there are provided a method for cell culture which comprises the step of culturing cells by using the aforementioned carrier for cell culture; a method for cell culture which comprises the step of obtaining a cell sheet by a solubilization treatment of the gel layer comprising an anionic polysaccharide and a polyvalent metal ion in the cell culture which is cultured by using the aforementioned carrier for cell culture; said method for cell culture, wherein the solubilization treatment is conducted by using a medium comprising a chelating agent and having a polyvalent metal ion concentration of 2.6 mM or less; and a cell sheet obtainable by the aforementioned method for cell culture.

From further aspects, there are provided a cell transfer method which comprises the step of further culturing the cell culture, which is obtainable by the method for cell culture comprising the step of culturing cells by using any one of the aforementioned carriers for cell culture, on another carrier for cell culture; a cell culture or a cell sheet obtainable by a solubilization treatment of the gel layer comprising an anionic polysaccharide and a polyvalent metal ion in the cell culture obtainable by said cell transfer method; said cell culture or the cell sheet, wherein the solubilization treatment is conducted by using a medium comprising a chelating agent and having a polyvalent metal ion concentration of 2.6 mM or less; a cell lamination method which comprises the step of culturing the cell culture obtainable by the aforementioned cell transfer method on another cultured cell; a cell lamination method which comprises the step of culturing the cell culture, which is cultured by using any one of the aforementioned carrier for cell culture, on another cultured cell; a laminated cell culture or a laminated cell sheet obtainable by the solubilization treatment of the gel layer comprising an anionic polysaccharide and a polyvalent metal ion in the cell culture obtainable by said cell lamination method; a physical reinforcing means which is provided on one side of one or more of the layers in a carrier for cell culture having multi-layer structure; and a medium for solubilization treatment of a gel layer comprising an anionic polysaccharide and a polyvalent metal ion in cell culture by using a carrier for cell culture comprising said layer, which comprises a chelating agent and having a polyvalent metal ion concentration of 2.6 mM or less.

### EFFECT OF THE INVENTION

The present invention provides a carrier for cell culture through which state of the cultured cells can be directly observed, and with which lamination of cell layers can be conducted easily.

### BRIEF EXPLANATION OF DRAWINGS

FIG. 1 shows a shape of the physical reinforcing means.

FIG. 2 shows the cell-adhesive gel layer in the carrier for cell culture used in Example 8.

### BEST MODE FOR CARRYING OUT THE INVENTION

The “carrier for cell culture” used in the specification means a structure which can be a carrier or a support in a process of cell culture. A form of the carrier for cell culture of the present invention is not particularly limited and is preferred to be a sheet form.

The carrier for cell culture of the present invention is characterized to comprise a water-containing polymer gel layer having a thickness of 0.01 μm or more and 5 μm or less. In the specification, a layer is sometimes referred to as a membrane.

“A water-containing polymer gel” means a hydrophilic polymer, and particularly means a water absorbing polymer which is insoluble in water but maintains water in the polymer so as to have a two- or three-dimensional support structure throughout the system. In the present invention, as a water-containing polymer gel layer, a layer is used which allows a diffusion of a substance such as a chelating agent in said layer to have the chelating agent move from one surface of said layer and reach to the other surface. Also in the present invention, as the water-containing polymer gel layer, a layer is used which does not allows a gel, comprising an anionic polysaccharide and a polyvalent metal ion such as alginic acid gel, to move from one surface of said layer to reach to the other surface. The water-containing polymer gel layer used in the present invention is not particularly limited as far as the layer has the above properties, and may be a synthetic polymer, a natural polymer, or a biopolymer. Examples of the water-containing polymer gel include acrylamide gel, bridged acrylic acid gel, agarose, gelatin, dextran, chitosan, silica gel and the like. Chitosan is preferred to be used.

In the carrier for cell culture of the present invention, the water-containing polymer gel layer is preferred to be a support. A support in the carrier for cell culture is meant herein a layer as a base plate for preparation of the carrier for cell culture having the multi-layer structure.

In the present invention, the water-containing polymer gel layer is characterized to have a thickness of 0.01 μm or more and 5 μm or less. The thickness is preferably 0.1 μm or more and 4 μm or less, and more preferably 0.5 μm or more and 3 μm or less. When the water-containing polymer gel layer is thinner than the above, a problem arises in that a membrane formation is insufficient and gives tears, fractures, or holes. When the water-containing polymer gel layer is thicker than the above, a problem arises in that adverse effects on cells are caused by a slow diffusion of medium components or a chelating agent in a delamination treatment.

A thickness of a layer mentioned in the specification indicates a thickness which is measured in a sufficiently dried state, unless otherwise specifically mentioned. The thickness of a layer is sometimes referred to as “dried membrane thickness”. Measurement of the thickness of a layer can be conducted by using a section image of electron microscope, a film thickness micrometer, an ellipsometer, an angle adjustable XPS, an optical interferometric film thickness meter and the like. Preferably, a film thickness micrometer, a section image of electron microscope, or an optical interferometric film thickness meter may be used.

The water-containing polymer gel layer of the carrier for cell culture of the present invention can be prepared by various methods for preparing a water-containing polymer gel membrane which are generally known. Examples of the methods include a method of casting a solution of a water-containing polymer gel (the casting method), a method of coating by using a barcoater (the barcoat method), and a method of coating by using a gapcoater (the gapcoat method). Among them, the barcoat method and the gapcoat method are preferred.

As a water-containing polymer gel used in the present invention, a gel which has a viscosity in a liquid state, which will be later defined in the specification, of 1,000 mPa•s or more and 50,000 mPa•s or less may be used. The viscosity is preferably 3,000 mPa•s or more and 30,000 mPa•s or less, and more preferably 6,000 mPa•s or more and 20,000 mPa•s or less.

A viscosity of a polymer solution prepared under the same condition can be used as an index of a molecular weight of the polymer. A higher value of the viscosity means a higher molecular weight. A viscosity of the water-containing polymer gel in a liquid state defined in the specification means a viscosity of a solution before gelation, in which said polymer 12 g is dissolved in 1000 g of 1 mass % acetic acid solution, measured by using a B-type viscosimeter at 25° C. When a polymer solution has a lower viscosity, i.e., a lower molecular weight, it is suggested that a water-containing polymer gel prepared has a weaker strength. Further, due to the lower viscosity, the solution flows out when a water-containing polymer gel layer is prepared, which leads to nonuniform thickness of a membrane; skinning phenomenon where only the most outer surface of the solution is dried to give a coat; and a prolonged period of time for drying the solution. When a polymer solution has a too high viscosity, i.e., a higher molecular weight, the polymer gel membrane prepared has a sufficient strength. However, the polymer has no casting property for preparation of a water-containing polymer gel membrane, which results in problems such as nonuniform thickness of the membrane or failure of formation of a coating membrane. By using a water-containing polymer gel having a viscosity within the aforementioned range, the water-containing polymer gel layer having a sufficiently high strength and a thickness as specified above can be obtained.

According to a preferred embodiment of the present invention, an example includes the carrier for cell culture wherein the water-containing polymer gel layer is adjacent to the gel layer comprising an anionic polysaccharide and a polyvalent metal ion. As the anionic polysaccharide, examples include alginic acid, dextran sulfate, carboxymethylcellulose, carboxymethyl dextran, hyaluronic acid, and the like. Preferably, alginic acid is used.

Alginic acid exists in nature as a cell wall-constituting polysaccharide or an intercellular filling substance of brown algae, and can be obtained from the algae as raw materials. Examples of the brown algae as a raw material include brown algae belonging to Order Fucales, Family Durvilleaceae, Genus Durvillea (e.g., D. potatorum), Order Fucales, Family Fucaceae, Genus Ascophyllum (e.g., A. nodosum), Order Laminariales, Family Laminariaceae, Genus Laminaria (e.g., Laminaria japonica, Laminaria longissima), Order Laminariales, Family Laminariaceae, Genus Eisenia (e.g., Eisenia bicyclis), Order Laminariales, Family Laminariaceae, Genus Ecklonia (e.g., Ecklonia cava, Ecklonia kurome), and Order Laminariales, Family Lessoniaceae, Genus Lessonia (e.g., L. flavikans). Commercially available alginic acid can also be used. A G/M ratio of alginic acid is not particularly limited. A larger G/M ratio provides higher gel formation ability, and accordingly, a larger G/M ratio is preferred. Specifically, the ratio may preferably be from 0.1 to 1, more preferably from 0.2 to 0.5.

The “alginic acid gel” means alginic acid gelled by a chelate structure formed with a carboxylic acid group in the molecule of alginic acid and a polyvalent metal ion, and “alginic acid gel layer” means alginic acid gel in the form of a layer. Alginic acid is a block copolymer consisting of gluronic acid (G) and mannuronic acid (M), and it is considered that the polyvalent metal cation enters into a pocket structure of the M block to form an egg box and thereby cause the gelation. Specific examples of the polyvalent metal cation that can cause the gelation of alginic acid include, for example, metal ions such as barium (Ba), lead (Pb), copper (Cu), strontium (Sr), cadmium (Cd), calcium (Ca), zinc (Zn), nickel (Ni), cobalt (Co), manganese (Mn), iron (Fe) and magnesium (Mg) ions. Among them, particularly preferred are calcium ion, magnesium ion, barium ion, and strontium ion. The “alginic acid gel” may be a polyion complex gel of alginic acid and an organic polymer compound having a cationic residue. Examples of the organic polymer compound having a cationic residue include compounds having two or more amino groups such as polylysine, chitosan, gelatin, and collagen.

The gelation of alginic acid may be achieved in a conventional manner. For example, the gelation of alginic acid can be carried out by using ion exchange. For example, when calcium ions are added to an aqueous solution of sodium alginate, ion exchange quickly occurs to give calcium alginate gel. More specifically, an 0.2 to 5% aqueous solution of sodium alginate may be applied on a water-containing polymer (for example chitosan) gel layer, which is then immersed in a 0.01 to 1.0 M aqueous solution of calcium chloride for soak with calcium chloride, and then left at 20 to 30° C. for 3 minutes to 3 hours. When a gelation of alginic acid is conducted by using a water-containing polymer as mentioned above, a carrier for cell culture comprising a water-containing polymer gel layer and an alginic acid gel layer formed thereon can be obtained.

The thickness of the gel layer comprising an anionic polysaccharide and a polyvalent metal ion of the carrier for cell culture of the present invention is preferably 0.01 μm or more and 50 μm or less, more preferably 0.1 μm or more and 10 μm or less, and further preferably 0.5 μm or more and 5 μm or less. When a solid content in the alginic acid gel layer is too low, a problem arises that a layer consisting of a satisfactory membrane cannot be formed to give a hole, whereas when too high, different problems of a curl or fracture of a dried membrane, deformation in a culture process, or insufficient solubilization in an alginic acid gel solubilization process may arise.

The cultured cell layer formed on the carrier for cell culture of the present invention can be delaminated as a cell sheet by a solubilization treatment of the gel layer comprising an anionic polysaccharide and a polyvalent metal ion. The cell sheet comprises the cultured cell layer, and preferably a cell adhesive gel layer which will be mentioned later. The solubilization treatment of the gel layer comprising an anionic polysaccharide and a polyvalent metal ion can be carried out by removing a cation component that constitutes the gel layer comprising an anionic polysaccharide and a polyvalent metal ion. When the cation is a polyvalent metal ion, the treatment can be conducted by subjecting the carrier for cell culture, on which a cultured cell layer is formed, to: 1) immersion in a medium added with an ion such as phosphate ion which forms a chelate or a poorly soluble salt with the polyvalent metal ion; 2) immersion in a medium added with an aqueous solution of a chelating agent; 3) immersion in a medium wherein polyvalent metal ions are reduced; or 4) masking of the polyvalent metal ion in the culture medium of said cell with a chelating agent. Generally, a medium for cell culture contains a lot amount of phosphate ions. Therefore, the solubilization treatment of the gel layer comprising an anionic polysaccharide and a polyvalent metal ion is preferably conducted by using a medium containing the polyvalent metal ion at a lower concentration than that of a minimum medium generally used for a cell culture and further comprising a chelating agent. Specifically, said concentration is preferably 2.6 mM or less, more preferably 3 μM or less, and further preferably 0.5 μM or less. Most preferably, the concentration is substantially zero. The concentration of the chelating agent is preferably 2.3 mM or more and 26,000 mM or less, and more preferably 2.3 mM or more and 2,600 mM or less. By using the medium wherein the polyvalent metal ions are reduced as mentioned above, a solubilization of the gel layer comprising an anionic polysaccharide and a polyvalent metal ion can be achieved with reduced invasion of a chelating agent into a cell.

Examples for the chelating agent used in the present invention include, for example, ethylenediamine-di-orthohydroxyphenylacetic acid, diaminopropanetetraacetic acid, nitrilotriacetic acid, hydroxyethylethylenediaminetriacetic acid, dihydroxyethylglycine, ethylenediaminediacetic acid, ethylenediaminedipropionic acid, iminodiacetic acid, diethylenetriaminepentaacetic acid, hydroxyethyliminodiacetic acid, 1,3-diaminopropanoltetraacetic acid, triethylenetetraminehexaacetic acid, trans-cyclohexanediaminetetraacetic acid, ethylenediaminetetraacetic acid (EDTA), glycol ether-diaminetetraacetic acid, O,O′-bis(2-aminoethyl)ethylene glycol-N,N,N′,N′-tetraacetic acid (EGTA), ethylenediaminetetrakismethylenephosphonic acid, diethylenetriaminepentamethylenephosphonic acid, nitrilotrimethylenephosphonic acid, 1-hydroxyethylidene-1,1-diphosphonic acid, 1,1-diphosphonoethane-2-carboxylic acid, 2-phosphonobutane-1,2,4-tricarboxylic acid, 1-hydroxy-1-phosphonopropane-1,3,3-tricarboxylic acid, catechol-3,5-disulfonic acid, sodium pyrophosphate, sodium tetrapolyphosphate, sodium hexametaphosphate, 1-hydroxypropylidene-1,1-diphosphonic acid, 1-aminoethylidene-1,1-diphosphonic acid, and salts thereof. Among them, particularly preferred examples include EDTA, EGTA, ethylenediaminetetraphosphonic acid, and 1-hydroxyethylidene-1,1-diphosphonic acid.

Further, in the medium for the solubilization treatment of the gel layer comprising an anionic polysaccharide and a polyvalent metal ion, a concentration of cationic amino acids is less than that of cationic amino acids in a minimum medium generally used for cell culture. Specifically, the concentration is preferably 1.0 mM or less, more preferably 2 μM or less, and further preferably 0.5 μM or less. Most preferably, the concentration is substantially zero. “Cationic amino acid” means L-Lysin(Lys), L-Arginine(Arg), L-Histidine(His), L-Cystine(Cys), or a salt thereof.

The solubilization treatment of the gel layer comprising an anionic polysaccharide and a polyvalent metal ion may be conducted by one or more immersions in any of the above mediums for the solubilization treatment. When two or more immersions are conducted, the mediums for the solubilization treatment may be the same or different.

For the solubilization treatment of the gel layer comprising an anionic polysaccharide and a polyvalent metal ion, i.e., for the immersion of the carrier for cell culture, on which a cultured cell layer is formed, in the medium for the solubilization treatment, the immersion may preferably be conducted so that a chelating agent infiltrate from the side of the water-containing polymer gel layer. By such operation, the water-containing polymer gel layer and the gel layer comprising an anionic polysaccharide and a polyvalent metal ion can be easily separated, and a cell sheet comprising cultured cell can be easily delaminated from the water-containing polymer gel layer. It is not necessary to completely remove the gel layer comprising an anionic polysaccharide and a polyvalent metal ion by the solubilization treatment of the gel layer comprising the anionic polysaccharide and the polyvalent metal ion, and the gel layer comprising the anionic polysaccharide and the polyvalent metal ion remained unsolubilized may be left on the cell sheet. However, the gel layer comprising an anionic polysaccharide and a polyvalent metal ion is preferably solubilized and removed as completely as possible.

According to another preferred embodiment of the present invention, an example includes the carrier for cell culture wherein the most outer surface of the side for cell culture is a cell-adhesive gel layer.

The “cell-adhesive gel layer” means a hydrogel in the form of a layer having a cell-adhesive property. The gel is not particularly limited, as long as the gel has no cytotoxicity and allows adhesion with cells under an ordinary culture condition, and any natural or synthetic compound can be used. A preferred example includes an extracellular matrix component gel in the form of a layer. The extracellular matrix is generally defined as “a stable biological structure existing extracellularly in an animal tissue and is a complex aggregate formed by biological polymers which are synthesized by cells, and secreted and accumulated outside the cells” (Dictionary of Biochemistry (3rd edition), p. 570, Tokyo Kagaku Dojin), and the matrix plays roles of materially supporting cells, regulating cellular activities (i.e., a role of transmitting extracellular information to a cell to change its activities) and the like.

The “extracellular matrix component” means a component of an extracellular matrix, and specific examples include collagen, elastin, proteoglycan, glucosaminoglycan (hyaluronic acid, chondroitin sulfate, dermatan sulfate, heparan sulfate, heparin, keratan sulfate, and the like), fibronectin, laminin, vitronectin, gelatin and the like. Particularly preferred examples among them are collagen, atelocollagen, Matrigel (gel consisting of type IV collagen, laminin, and heparan sulfate), hyaluronic acid, and gelatin. The extracellular matrix component can be obtained in a conventional manner, and commercially available extracellular matrix components may also be used. The cell adhesion component can be gelled in a conventional manner. For example, when the cell adhesion component is collagen, a collagen gel can be obtained by incubating a 0.3 to 0.5% aqueous solution of collagen at 37° C. for 10 to 20 minutes. A gelling agent may be used for the gelation of the extracellular matrix component, if needed.

A thickness of the cell-adhesive gel layer according to the present invention may preferably be 0.005 μm or more and 5.0 μm or less, more preferably 0.005 μm or more and 1.0 μm or less, further preferably 0.005 μm or more and 0.5 μm or less. When the cell-adhesive gel layer is too thick, a crack may be generated on the layer during a drying process, and further a cell transfer may become significantly difficult.

When the cell-adhesive gel layer is formed on the gel layer comprising an anionic polysaccharide and a polyvalent metal ion, the gel layer comprising an anionic polysaccharide and a polyvalent metal ion and the cell-adhesive gel layer may be prepared separately and then laminated. Preferably, a solution containing cell-adhesive component may be added on the gel layer comprising an anionic polysaccharide and a polyvalent metal ion and then the solution is subjected to gellation. The cell-adhesive gel layer does not have enough physical strength for lamination or delamination, therefore it is difficult to delaminate the cell-adhesive gel layer from a container (for example, dish, petri dish or the like) on which the cell-adhesive gel layer is formed. The cell-adhesive gel layer as a ultrathin layer can be easily obtained by immersing the gel layer comprising an anionic polysaccharide and a polyvalent metal ion in a solution containing cell-adhesive components (the immersion method), by coating (the coating method), or by casting (the casting method). Any of the above methods can be used for the preparation of the carrier for cell culture of the present invention. Among them, the casting method is preferably used. For example, when a collagen gel layer is formed on alginic acid gel layer, a commercially available 0.3 to 0.5% aqueous solution of collagen may be diluted to a suitable concentration if necessary, then the resulting solution may be casted on the calcium alginate gel prepared by the above method and dried to obtain a collagen gel layer formed on alginic acid gel layer.

The cultured cell layer on the carrier for cell culture of the present invention can be delaminated as a cell sheet by the solubilization treatment of the gel layer comprising an anionic polysaccharide and a polyvalent metal ion as mentioned above. A physical reinforcing means can be provided on the cell-adhesive gel layer surface at the opposite side of the water-containing polymer gel layer to improve operability during the solubilization treatment. A material for the physical reinforcing means is not particularly limited so long as the material has no effect on cells. Examples include metals (for example, iron, stainless-steel, titanium, gold, and the like), plastics (for example, polystyrene, polycarbonate, polyethylene, polypropylene, acrylic plastic, and the like), inorganic materials such as ceramics. Preferred examples include stainless-steel, titanium, and plastics. The physical reinforcing means may be in any form so long as the means can improve operability of the carrier for cell culture of the present invention. The physical reinforcing means may preferably in a plate form, and a thickness thereof is preferably 0.1 μm or more and 10 mm or less, more preferably 1 μm or more and 1 mm or less, further preferably 10 μm or more and 20 μm or less.

The physical reinforcing means may be in any shape as long that the means contains a part through which cells cultured can be observed. Examples of the shapes include a circle, a polygon (triangle, quadrilateral, hexagon, or the like), and the combination thereof (sector or the like). Among them, a circle-like shape is preferred. One or more of the parts through which the cells cultured can be observed may be present. Further, the physical reinforcing means preferably may preferably has an asymmetric shape so that the side of the water-containing polymer gel layer attached with the physical reinforcing means can be easily identified.

The physical reinforcing means may be attached to the water-containing polymer gel membrane by any method so long as a cell culture is not affected. For example, attachment may be conducted by a method using a commercially available adhesive agent (for example, Aronalpha (Krazy Glue), Bond (glue)) after preparation of a water-containing polymer gel membrane, or putting the physical reinforcing means on a water-containing polymer gel membrane under an undried condition.

Depending on a type of a material, the physical reinforcing means may sometimes have a sharp edge. It is concerned that the physical reinforcing means might break the water-containing polymer gel with its sharp edge, or might harm an operator. Therefore, the physical reinforcing means is preferred to have no sharp edge. The sharp edge can be removed by any method as long as the method causes no problem on cell culture. Examples of the method include physical polish (for example, polish by using a file or the like) and a chemical treatment (for example chemical etching or the like). According to the present invention, when the physical reinforcing means is made of stainless steel, the means is preferably subjected to a chemical treatment such as chemical etching.

To improve operability for the delamination as a cell sheet of a cultured cell layer formed on the carrier for cell culture of the present invention, the carrier for cell culture having a cell-adhesive gel layer on the most outer surface for cell culture may be provided with a part unmodified with the cell-adhesive gel layer, i.e., a part wherein the cell-adhesive gel layer is not formed, on the most outer surface for cell culture. The part unmodified with the cell-adhesive gel layer is consequently formed with the water-containing polymer gel layer or the gel layer comprising an anionic polysaccharide and a polyvalent metal ion. By providing the part unmodified with the cell-adhesive gel layer, an operability for the delamination of the water-containing polymer gel layer from the cell-adhesive gel layer can be improved. By pinching the water-containing polymer gel layer in the part unmodified with the cell-adhesive gel layer with tweezers or the like, the water-containing polymer gel layer can be removed without touch to the cell-adhesive gel layer, thereby a reduced harmful effect on cells is achieved. The part unmodified with the cell-adhesive gel layer may preferably be provided at a corner of the water-containing polymer gel layer of the carrier for cell culture of the present invention.

As a method for providing the part unmodified with the cell-adhesive gel layer, any method can be used so long as the method causes no problem in cell culture. An example includes a masking method which is generally well known. In the method, a part on a water-containing polymer gel layer to be unmodified with the cell-adhesive gel layer is covered beforehand with another material, and then the water-containing polymer gel layer is modified with cell-adhesive gel components and further the material which covers said part is removed to obtain the part unmodified with the cell-adhesive gel layer.

A type of the material used as the coverage to provide the part unmodified with the cell-adhesive gel layer is not limited so long as the material causes no problem in cell culture. Examples include silicon rubber, masking tape or adhesive tape which is commercially available, plastics (for example, polystyrene, polycarbonate, polyethylene, polypropylene, acrylic plastic, and the like), metals (for example, iron, stainless-steel, titanium, gold, and the like), and silicon rubber or commercially available masking tape is preferable.

A shape of the part unmodified with the cell-adhesive gel layer is not limited so long as the shape causes no problem in cell culture, and a circle, a polygon (triangle, quadrilateral, hexagon, or the like), or the combination thereof (sector or the like) is preferable. Triangle or sector is more preferable. An area of the part unmodified with the cell-adhesive gel layer is not limited so long as the area causes no problem in cell culture, and preferably diameter of 0.1 mm or more and 10 mm or less, more preferably diameter of 1 mm or more and 5 mm or less in terms of a circle.

As types of cells that can be cultured by using the carrier for cell culture of the present invention, specific examples include, fibroblasts, vascular endothelial cells, chondrocytes, hepatocytes, small intestine epithelial cells, epidermal keratinocytes, osteoblasts, bone marrow mesenchymal cells and the like, and a preferable example includes fibroblasts. For culture of cells, a culture medium (for example, D-MEM medium, MEM medium, HamF12 medium, or HamF10 medium) containing cells at a density of from 10,000 to 15,000 cells/ml is usually added onto the cell-adhesive gel layer. A condition for cell culture can be appropriately chosen depending on the type of cells to be cultured. When cells are cultured on the cell-adhesive gel layer, the culture may preferably be continued generally until a confluent cell monolayer is formed on the cell-adhesive gel layer.

Culture of cells using the carrier for cell culture of the present invention can be performed specifically as follows. The carrier for cell culture is placed inside a petri dish or the like, then an appropriate culture medium (for example, D-MEM medium, MEM medium, HamF12 medium, HamF10 medium) is added to the petri dish to immerse the carrier for 5 minutes, and then the medium is exchanged. After this procedure is repeated three times, the culture system is left for 12 to 24 hours so that the culture medium can infiltrate into the carrier for cell culture. Then, the culture medium in the petri dish is discarded, and then cells are inoculated onto the cell-adhesive gel layer of the carrier for cell culture, and further an appropriate culture medium (for example, D-MEM medium, MEM medium, HamF12 medium, HamF10 medium) is added to the petri dish. After the system is left at 37° C. for 1 to 2 hours so that the cells can be held by (adhered to) the cell-adhesive gel layer, the culture is continued at 37° C. During the culture, the culture medium may be exchanged, if needed. Usually, the culture medium is exchanged every 0.5 to 2 days of the culture.

A cell culture obtained by culturing cells using the carrier for cell culture of the present invention contains the carrier for cell culture of the present invention and a cell layer held by said carrier for cell culture. The cell layer held by the carrier for cell culture is preferably a cell layer formed on the cell-adhesive gel layer.

The cell sheet, which is obtained by subjecting the gel layer comprising an anionic polysaccharide and a polyvalent metal ion gel layer to the solubilization treatment, contains a cell layer, and accordingly can be used for lamination and transfer of cell layers. For lamination of cell layers, a cell culture cultured by using the carrier for cell culture of the present invention may be laminated on cells cultured beforehand with or without applying load, which may be further cultured, then the gel layer comprising an anionic polysaccharide and a polyvalent metal ion may be solubilized. Alternatively, each of cell sheets obtained by solubilizing the gel layers comprising an anionic polysaccharide and a polyvalent metal ion may be laminated, or a cell sheet obtained by solubilizing the gel layer comprising an anionic polysaccharide and a polyvalent metal ion may be laminated on a separately prepared cell culture. Further, a cell sheet or cell culture containing the cell layers laminated by the above method or the like may be laminated on a separately prepared cell layer. The separately prepared cell layer may be the cell layer of the cell culture which is cultured by using the carrier for cell culture of the present invention, or may be the cell layer of the cell culture which is cultured by using another carrier for cell culture, or a cell sheet. Kinds of cells of the cell layers to be laminated may be the same or different. The number of the cell layers to be laminated is not particularly limited. Generally, the number is from 1 to 10, preferably from 1 to 5, more preferably from 1 to 3.

For transfer of a cell layer, the cell culture which is cultured by using the carrier for cell culture of the present invention may be loaded on another substrate for cell culture with or without applying load, which may be further cultured, then the gel layer comprising an anionic polysaccharide and a polyvalent metal ion may be solubilized. Alternatively, a cell sheet obtained by solubilizing the gel layer comprising an anionic polysaccharide and a polyvalent metal ion may be transferred to another substrate. The cell culture to be transferred may be a laminated cell culture.

Examples of preferred method for the lamination or the transfer include a method of culturing the cell culture, which is cultured by using the carrier for cell culture of the present invention, on cells cultured beforehand or cultured on another substrate for cell culture, and then dissolving the gel layer comprising an anionic polysaccharide and a polyvalent metal ion.

The method for cell culture with applying load may be any method so long as sufficient load is applied so as not to generate unevenness in cells or a substrate on which cells are transferred. If cells are sealed by applying load, cells may be smothered. Therefore, at least either of the cell culture substrates to be transferred or that to receive the transfer preferably consists of a water-permeable gel, a water-containing polymer gel, or a combination thereof. Further, for transfer avoiding unevenness, the load should be applied so as to sufficiently cover the surface of the cells. However, uniform contact may disturb diffusion of oxygen, and therefore, the load may preferably be applied through nonwoven fabric (nylon, polyester, stainless steel and the like) or the like so as not to disturb diffusion of oxygen.

In the cell culture method with applying load, the load to be applied is preferably from about 0.1 g/cm² or more and 50 g/cm² or less, more preferably from about 0.50 g/cm² or more and to 10 g/cm² or less. A culture period of time of the cells applied with load is not particularly limited, and can be appropriately chosen so that sufficient transfer of cells can be achieved. The period of time is preferably 4 hours or more and 72 hours or less, more preferably from 6 hours or more and to 48 hours or less. A cell culture without applying load is preferred in the present invention.

When the carrier for cell culture of the present invention is prepared, a solution for preparation containing a carbodiimide may be used to improve adhesiveness. A carbodiimide and N-hydroxysuccinimide may be added to the solution for preparation of any layer, and preferably, added to a solution for preparation of the gel layer comprising an anionic polysaccharide and a polyvalent metal ion or added in the water-containing polymer gel for immersion, or alternatively, added for immersion in a solution in which calcium chloride is co-dissolved after the application of the gel layer comprising an anionic polysaccharide and a polyvalent metal ion. A class of water-soluble carbodiimide is preferred. An example include 1-ethyl-3-(3-dimethylaminopropyl)-carbodiimide hydrochloride. When the carbodiimide is used, a concentration is preferably 0.01 mg/l or more and 200 g/l or less. N-hydroxysuccinimide may be used as a catalyst, and the concentration is preferably 1 mass % or more and 50 mass % or less.

In the present invention, preferably a carbodiimide is not used unless specifically needed.

The carrier for cell culture of the present invention may be sterilized by any method. Sterilization by radiation such as electron beam, γ-ray, X-ray, and ultraviolet ray may preferably be used. An electron beam, γ-ray, and ultraviolet ray may more preferably be used, and electron beam sterilization may be particularly preferred. An exposure dose for the electron beam sterilization is preferably from 0.1 kGy or more and 65 kGy or less, most preferably 1 kGy or more and 40 kGy or less. Chemical sterilization such as ethylene oxide gas (EOG) sterilization and sterilization using a high temperature such as high pressure steamy gas sterilization may not be preferred, because the cell-adhesive layer and the gel layer comprising an anionic polysaccharide and a polyvalent metal ion may be decomposed. A carrier for cell culture sterilized as described above can be stored at room temperature for a long period of time, if it is stored under a sterile condition. The aforementioned sterilization methods may be used each alone or in combination. The same sterilization method may be applied repeatedly.

When a vascular endothelial cell layer or hepatocyte layer, for example, is used as a cell layer to be laminated, a three-dimensional tissue structure of liver can be constituted. This three-dimensional tissue structure can be applied to in vitro drug permeability test, and also to an alternative model for animal experiment or to organs for transplantation. The laminated cell layers can be cultured under culture conditions depending on the kinds of cells constituting the cell layers. For the culture, various mediums such as D-MEM medium, MEM medium, HamF12 medium, and HamF10 medium can be used.

### EXAMPLES

The present invention will be more specifically explained by referring to the following examples. However the scope of the present invention is not limited to these examples.

### Example 1

### Preparation of a Water-Containing Polymer Gel Membrane

(1) Preparation of Water-Containing Chitosan Gel Membrane A (A-1 to A-4)

Chitosan CT-1000 (produced by Wako Pure Chemical Industries) (12 g) was gradually added to 1 mass % acetic acid solution (1,000 g) and stirred at 40° C. for 3 hours for dissolution. The solution was filtrated by microfilter FG-30 produced by Fuji Photo Film Co., Ltd. The filtrated acetic acid solution of chitosan was applied to a polyethylene terephthalate film (film thickness: 185 μm) which was prepared beforehand by an applicator with dried membrane thickness shown in Table 1, and dried at 40° C. for 5 hours.

The dried chitosan membrane was immersed in a 10 mass % methanol solution of sodium hydroxide for 60 minutes, subsequently in PBS (Dulbecco's phosphate-buffered saline) for 60 minutes. The membrane was then washed with running water for 60 minutes to obtain a chitosan gel. The above obtained chitosan gel membrane was dried at 32° C. overnight, and stored in a plastic bag at room temperature.

(2) Preparation of Water-Containing Chitosan Gel Membrane B (B-1 to B-4)

Each of chitosan CT-10, 100, 500, and 1000 (produced by Wako Pure Chemical Industries) (12 g) was gradually added to 1 mass % acetic acid solution (1000 g) and stirred at 40° C. for 3 hours for dissolution. The solution was filtrated by microfilter FG-30 produced by Fuji Photo Film Co., Ltd. The filtrated acetic acid solution of chitosan was applied to a polyethylene terephthalate film (film thickness: 185 μm) which was prepared beforehand by an applicator with dried membrane thickness of 1 μm and dried at 40° C. for 3 hours

(3) Evaluation of the Water-Containing Polymer Gel Membrane

The water-containing polymer gel membranes obtained in the above (1) and (2) were evaluated as follows.

(A) Strength of Wet Membrane

Strength of the membranes after immersing the dried chitosan membranes in distilled water at 37° C. for 3 hours was evaluated. Evaluation of the strength of the membranes was conducted using the following criteria, wherein ◯ indicates an acceptable result.
Membrane that can be pinched with tweezers without any tear and has practically no problem . . . ◯
Membrane that can be pinched with tweezers sometimes with tears . . . Δ
Membrane that is in jelly-like form and cannot be pinched with tweezers . . . ×
   (B) Diffusion Property

Each of the dried chitosan membranes was placed in an isostatic dialysis cell (produced by Sanplatec) having a cavity at each side of the membrane. An aqueous solution of a chelating agent was added to the cavity of one side, and distilled water was added to the cavity of the other side at 37° C. The side of the distilled water was sampled with passage of time and the amount of the chelating agent penetrated was quantified to evaluate diffusion of substances in the membrane. Evaluation of the diffusion property was conducted using the following criteria, wherein ◯ indicates an acceptable result.
Amount of the chelating agent penetrated in 30 minutes is 80% or more of the aqueous solution of the chelating agent . . . ◯
Amount of the chelating agent penetrated in 30 minutes is 20% or more and less than 80% of the aqueous solution of the chelating agent . . . Δ
Amount of the chelating agent penetrated in 30 minutes is less than 20% of the aqueous solution of the chelating agent . . . ×
   (C) Viscosity

Viscosity of the acetic acid solution of chitosan was measured by using a B-type viscosimeter at 25° C.

Results are shown in Table 1 and Table 2.

The water-containing polymer gel gave good results in the range according to the present invention.

TABLE 1
Properties of chitosan membrane

| | | Dried | | | |
|---|---|---|---|---|---|
| | | membrane | Wet | | |
| Sample | Viscosity | thickness | membrane | Diffusion | |
| name | (Pa · s) | (μm) | strength | property | |
| A-1 | 9200 | 0.001 | X | — (*1) | Comparative |
| | | | | | example |
| A-2 | 9200 | 1 | ◯ | ◯ | Present |
| | | | | | invention |
| A-3 | 9200 | 2 | ◯ | ◯ | Present |
| | | | | | invention |
| A-4 | 9200 | 10 | ◯ | X | Comparative |
| | | | | | example |

*1: No data was obtained because the membrane was thin to give a tear and not able to be placed in the dialysis cell.

TABLE 2
Properties of chitosan membrane

| Sample | Type of | Viscosity | Wet membrane | |
|---|---|---|---|---|
| name | chitosan | (Pa · s) | strength | |
| B-1 | CT-10 | 380 | X | Comparative example |
| B-2 | CT-100 | 810 | X | Comparative example |
| B-3 | CT-500 | 3500 | ◯ | Present invention |
| B-4 | CT-1000 | 9200 | ◯ | Present invention |

### Example 2

### Preparation of a Carrier for Cell Culture

(1) Preparation of Water-Containing Chitosan Gel Membrane/Calcium Alginate Gel Laminated Membrane

On each of the water-containing chitosan gel membranes A and B obtained in (1) and (2) of Example 1, 2 mass % aqueous sodium alginate solution was coated so as to give a thickness of 500 μm of the wet coat membrane. The coated membrane was immersed in a 0.25M solution of calcium chloride in 25% methanol solution for 60 minutes, and washed with running water for 30 minutes to obtain water-containing chitosan gel/calcium alginate gel laminated membranes AA and BB. The thickness of the dried alginic acid gel membranes was 10 μm measured from the electron microscopic image.

The similar samples were successfully obtained having thickness of 100, 300, and 1000 μm of the wet coat membrane of the aqueous sodium alginate solution.

(2) Modification with Collagen

On the water-containing chitosan gel/calcium alginate gel laminated membranes AA and BB which were obtained in the above (1) and were not dried, a three times-diluted aqueous solution of Cellmatrix I-P (produced by Nitta Gelatin) was casted, and the coated membranes were dried to obtain a ultrathin-collagen-layer modified membranes AA-11 to 13, and BB-11 to 13 (Table 3).

TABLE 3
Carrier for cell culture

| Sample | Chitosan | Thickness of the wet | |
|---|---|---|---|
| number | membrane | alginic acid coat | |
| AA-11 | A-1 in Table 1 | 500 μm | Comparative example |
| AA-12 | A-2 in Table 1 | ″ | Present invention |
| AA-13 | A-3 in Table 1 | ″ | Present invention |
| AA-14 | A-4 in Table 1 | ″ | Comparative example |
| BB-11 | B-1 in Table 2 | ″ | Comparative example |
| BB-12 | B-2 in Table 2 | ″ | Comparative example |
| BB-13 | B-3 in Table 2 | ″ | Present invention |
| BB-14 | B-4 in Table 2 | ″ | Present invention |

### Example 3

### Sterilization

The membranes obtained in Example 1 were subjected to UV sterilization for 1, 2, and 3 hours and electron beam sterilization at 20, 40, 60, 80, and 100 kGy. As a result, no bacterium was found in each of the membranes. In the samples not subjected to any sterilization treatment, 8,600 cells/m² of bacteria were observed.

### Example 4

### Culture of Cells by Using the Carrier for Cell Culture

Cells were cultured by using the carrier for cell culture as follows.

(1) Used Cell

CHL (Chinese Hamster Lung Cell)

(2) Used Medium

Eagle's minimum medium containing 10% fetal bovine serum

(3) Carrier for Cell Culture

The carrier for cell culture of the present invention prepared in Example 1 was placed on the bottom of a petri dish for cell culture made of polystyrene so that the cell adhesive layer became an upper surface. A petri dish for cell culture made of polystyrene was prepared separately as a comparative example. These samples were subjected to UV sterilization or electron beam sterilization and added with the medium for immersion for 5 minutes. This procedure was repeated 3 times. The samples were left for two overnights to allow the medium to infiltrate into the carrier.

(4) Inoculation of Cells

The cells cultured beforehand were collected by trypsin treatment, and the cell density was adjusted to 42,000 cells/ml. After each medium in the cells and dishes was discarded, the cell suspension was inoculated in the dishes at a cell number of 7,500 cells/cm², and then the medium was added.

(5) Culture

The cells were cultured at 37° C. for three days by using a CO₂ incubator.

(6) Results

The sample wherein the carrier for cell culture of the present invention prepared in Example 1 was placed on the bottom of a petri dish for cell culture made of polystyrene had transparency, thereby the growth state of the cultured cell was detailedly observable. The sample caused no problem in cell adhesion and gave no toxicity, which was almost in the same condition as that of the sample consisting solely of petri dish for cell culture made of polystyrene.

Further, in the sample of the carrier for cell culture of the present invention, no delamination of the cell-adhesive gel layer from the water-containing polymer gel was observed and no problem was caused.

Similar results were obtained when the cell was changed to HEPG2 (human hepatoma cell) or BAE (Bovine Aortic Endothelial Cell).

### Example 5

### Delamination and Transfer of a Cell Sheet

A sample, obtained by culturing the sample prepared in Example 2 in a similar manner to that of Example 4, was immersed in the following two types of medium for solubilization treatment at 37° C. for 10 minutes. Then a progress of the delamination of the cell-adhesive gel layer from the carrier for cell culture was observed. Subsequently, the delaminated cell sheet was placed on a petri dish for cell culture made of polystyrene and added with the medium, which was then cultured at 37° C. for three days by using a CO₂ incubator and observed under an optical microscope.

Results are shown in Table 4.

<Medium for Solubilization Treatment>

(a) Eagle minimum medium added with 1-hydroxyethylidene-1,1-diphosphonic acid (2.6 mM)
(b) A medium prepared by removing calcium ion and magnesium ion, and Lys, Arg, and His as cationic amino acids from medium (a)
   <Evaluation Criteria of Delamination Property> ⊚ and ◯ Indicate Acceptable Results.
⊚: The cell-adhesive gel layer delaminates from the water-containing polymer gel layer spontaneously in a floating manner.
◯: The cell-adhesive gel layer can be delaminated from the water-containing polymer gel layer easily by pinching the cell-adhesive gel layer with tweezers.
Δ: The cell-adhesive gel layer can be delaminated from the water-containing polymer gel layer by pinching the cell-adhesive gel layer with tweezers. However, a non-delaminated part remains.
×: The cell-adhesive gel layer fails to delaminate from the water-containing polymer gel layer even by pinching the cell-adhesive gel layer with tweezers.
   <Evaluation Criteria of Cell Survival Rate> ⊚ and ◯ Indicate Acceptable Results.
⊚: 90% or more cells survive
◯: 70% or more cells survive
Δ: 40% or more cells survive

×: Surviving cells account for 40% or less

TABLE 4

| | AA-11 | AA-12 | AA-13 | AA-14 |
|---|---|---|---|---|
| | (Comparative | (Present | (Present | (Comparative |
| | example) | invention) | invention) | example) |
| | Delamination | Delamination | Delamination | Delamination |
| | property/cell | property/cell | property/cell | property/cell |
| | survival rate | survival rate | survival rate | survival rate |
| (a) | — *3 | ◯/◯ | ◯/◯ | X/◯ |
| (b) | — *3 | ⊚/⊚ | ⊚/⊚ | X/⊚ |

*3: No data was obtained because the water-containing polymer gel layer was not treatable as a membrane and the sample was not able to be prepared

In the range of the present invention, excellent delamination property and high cell survival rate after the delamination were achieved simultaneously.

### Example 6

### Culture by Lamination of Cell Layers

In petri dishes for cell culture made of polystyrene applied with three-hour UV sterilization, the following cells were cultured in a similar manner to that of Example 4.
CHL (Chinese Hamster Lung Cell)
BRL (Buffalo Rat Liver 3A, ATCC No.: CRL 1442)
BAE (Bovine Aortic Endothelial Cell)

On the cells of the above three types cultured on the petri dishes, cells were transferred by using the medium for solubilization treatment (b) in Example 5 in a similar manner to that in Example 5 to obtain laminated cells. These laminated cells were cultured in the medium for 4 days. Then the states of the cells were observed under an optical microscope by staining with trypan blue. It was found that all kinds of the laminated cells were well cultured in the range of the present invention.

### Example 7

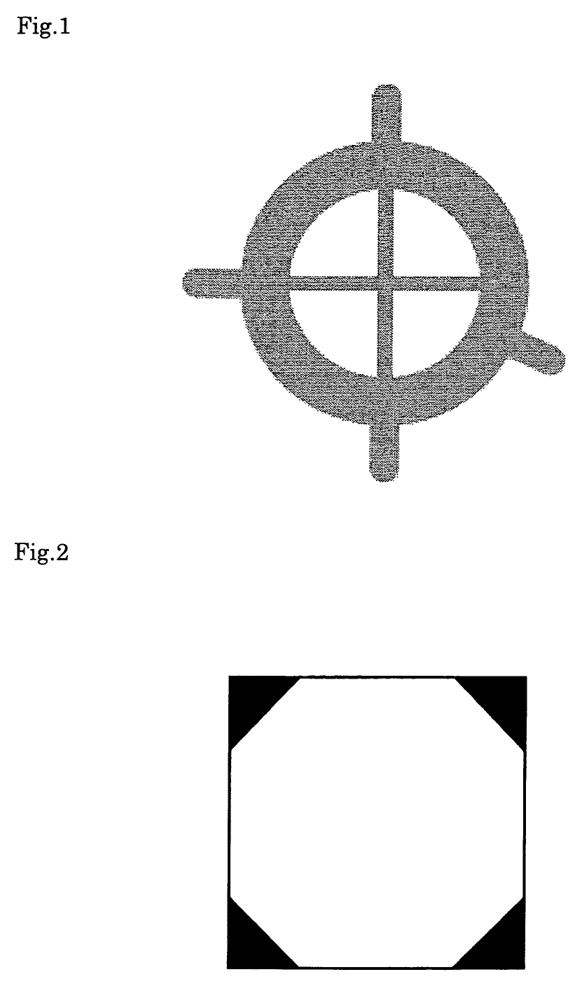

Except that a physical reinforcing means made of stainless steel shown in FIG. 1 was placed on chitosan soon after its application, 0.1 μm thick chitosan membrane was prepared in a similar manner to that of Example 1. The physical reinforcing means made of stainless steel used was 90 μm in thickness, 15 mm in inside diameter, and 20 mm in outside diameter and applied with chemical etching treatment.

The condition of the prepared chitosan membrane at separation from the polyethylene terephthalate film (PET) was evaluated as operability. The membrane provided with the physical reinforcing means was successfully separated from the PET film while maintaining the membrane form without any break or without forming any crinkle of the 0.1 μm chitosan membrane. Cell culture was conducted in a similar manner to those of Example 3, 4, 5, and 6, and satisfactory results were obtained in the range of the present invention.

### Example 8

### Preparation of Carrier for Cell Culture Having Parts Unmodified with Collagen

Except by using the chitosan sample membrane of A-2 in Example 1 and applying masking sheets (silicon rubber) in a isosceles right triangle shape having two 3 mm sides to the chitosan sample membrane with 12 mm intervals, calcium alginate layer and collagen layer were prepared in a similar manner to that of Example 2.

After drying, the laminated membrane prepared was cut and the masking sheets were removed to obtain a carrier for cell culture D (FIG. 2) having parts unmodified with collagen. The black parts in FIG. 2 (each isosceles right triangle having two 3 mm sides) are the parts unmodified with collagen.

By using the carrier for cell culture D, cell culture and delamination were carried out in a similar manner to those of Example 3, 4, and 5, and the delamination properties were evaluated. As a result, in the carrier for cell culture D having the parts unmodified with collagen, chitosan layer was easily delaminated with tweezers without touching the collagen layer in the delamination.

### Example 9

### Cell Culture, Transfer and Lamination of Cell Layer, and Delamination of the Water-Containing Polymer Gel Layer

(1) Preparation of a Water-Containing Chitosan Gel Membrane S (S1 to S7)

DAICHITOSAN 100D (produced by Dainichiseika Industries) (6 g) was gradually added to 1 mass % acetic acid solution (500 g) and stirred at room temperature for 7 hours for dissolution. The solution was filtrated by microfilter FG-30 produced by Fuji Photo Film Co., Ltd. The filtrated acetic acid solution of chitosan was applied to a polyethylene terephthalate film (length: 20 cm, width: 18 cm, film thickness: 195 μm) by an applicator with dried membrane thickness shown in Table 5, and dried at 37° C. overnight. The obtained membrane was immersed in a 1.9 mass % methanol solution of sodium hydroxide for 30 minutes, subsequently in PBS (Dulbecco's phosphate-buffered saline) for 30 minutes. The membrane was then immersed in distilled water bath for 30 minutes to obtain a chitosan gel membrane. The above obtained chitosan gel membrane was dried at room temperature overnight, and stored in a plastic bag

Chitosan gel membranes were prepared in a similar manner using each of DAICHITOSAN H and M produced by Dainichiseika Industries and KIMICA CHITOSAN H and B produced by KIMICA Company instead of DAICHITOSAN 100 D.

TABLE 5

| | | Dried | |
|---|---|---|---|
| | | membrane | |
| Sample | | thickness | |
| number | Type of chitosan | (μm) | |
| S1 | DAICHITOSAN 100D | 1 | Present invention |
| S2 | ″ | 2 | ″ |
| S3 | ″ | 4 | ″ |
| S4 | ″ | 8 | Comparative example |
| S5 | ″ | 12 | ″ |
| S6 | DAICHITOSAN H | 2 | Present invention |
| S7 | KIMICA | 2 | ″ |
| | CHITOSAN H | | |

(2) Preparation of Water-Containing Chitosan Gel/Calcium Alginate Laminated Membrane

On the chitosan gel membrane obtained in the above (1), 2 mass % aqueous KIMICA alginic B-1 solution produced by KIMICA Company was applied so as to give thickness of 300 μm of wet coat membrane. The coated membrane was immersed in a bath of 25% methanol solution containing calcium chloride at each concentration shown in Table 6 for 10 minutes, and immersed in distilled water bath for 30 minutes to obtain water-containing chitosan gel/calcium alginate gel laminated membrane. The thickness of the dried alginic acid gel membrane was measured to be 6 μm by using a film thickness meter.

Water-containing chitosan gel/calcium alginate laminated membranes having different dried membrane thicknesses of the alginic acid gel membrane were prepared by changing the thickness of wet coat membrane of the aqueous sodium alginate solution to 100, 500, and 1,000 μm.

(3) Preparation of Water-Containing Chitosan Gel/Calcium Alginate Laminated/Collagen Laminated Membrane

On the water-containing chitosan gel/calcium alginate gel laminated membrane obtained in the above (2), which were not dried, silicon rubber and a frame made of aluminium metal (inside length: 12 cm, inside width: 7 cm, and thickness: 5 mm) were placed. A 10-times concentrated Ham's F-12 medium (1 ml) was added to Cellmatrix I-P (produced by Nitta Gelatin) (8 ml), and the suspension was stirred under ice-cooling for one minute. The mixture was further added with a buffer solution (NaHCO₃ 2.2 g or 4.7 g was dissolved in 100 ml of 0.05 N NaOH aqueous solution) (1 ml) under ice-cooling and mixed so as not to make a foam. The solution (6.5 ml) was casted in the frame, which was then dried at the room temperature overnight, washed with distilled water, and dried again to obtain a water-containing chitosan gel/calcium alginate laminated/ultrathin collagen modified membrane (V1 to V10) (Table 6).

TABLE 6

| | | Thickness | | |
|---|---|---|---|---|
| | | of the | Calcium | |
| | | alginic | chloride | |
| | | acid coated | concen- | |
| Sample | Chitosan | membrane | tration | |
| number | membrane | (μm) | (mol/L) | |
| V1 | S2 | 300 | 0.02 | Present invention |
| V2 | ″ | ″ | 0.1 | ″ |
| V3 | ″ | ″ | 0.5 | ″ |
| V4 | S4 | ″ | 0.02 | Comparative example |
| V5 | ″ | ″ | 0.1 | ″ |
| V6 | S6 | ″ | 0.02 | Present invention |
| V7 | S7 | ″ | 0.02 | ″ |
| V8 | S2 | 100 | 0.05 | ″ |
| V9 | ″ | 500 | 0.05 | ″ |
| V10 | ″ | 1000 | 0.05 | ″ |

(4) By Using the Water-Containing Chitosan Gel/Calcium Alginate Laminated/Ultrathin Collagen Modified Membranes (V1 to V10) as Carrier for Cell Culture, which were Subjected to UV Sterilization for Two Hours, Cells were Cultured.
(a) Used Cell

BAE (Bovine Aortic Endothelial Cell)

(b) Used Medium

Eagle's minimum medium containing 10% fetal bovine serum

(c) Pretreatment

The water-containing chitosan gel/calcium alginate laminated/collagen modified membranes sterilized as mentioned above was placed on a support made of PET so that the cell adhesive layer became upper side. The resulting membrane including the support was then put on the bottom of a petri dish for cell culture made of polystyrene. The medium was added for immersion for 5 minutes and then the medium was exchanged. This procedure was repeated 3 times.

(d) Inoculation of Cells

The cells cultured beforehand were collected by trypsin treatment, and the cell density was adjusted to 40,000 cells/ml. After each medium in the cells and dishes was discarded, the cell suspension was inoculated into the dishes so as to give a cell number of 7,000 cells/cm², and then the medium was added.

(e) Culture

The cells were cultured at 37° C. for three days by using a CO₂ incubator.

(f) Results

The sample wherein the water-containing chitosan gel/calcium alginate laminated/collagen laminated membrane was placed on a support made of PET, which was further placed on the bottom of a petri dish for cell culture made of polystyrene, had satisfactory transparency, thereby the growth state of the cultured cell was detailedly observable. The sample caused no problem in cell adhesion and gave no toxicity, which was almost the same condition as that of the sample wherein the petri dish for cell culture made of polystyrene was solely used.

Similar results were obtained when cell was changed to HEPG2 (human hepatoma cell) or CHL (Chinese Hamster Lung Cell).

(5) Transfer and Lamination of Cell Layers, and Delamination of the Water-Containing Chitosan Gel Layer

For lamination of cell layers, the water-containing chitosan gel/calcium alginate laminated/collagen layer modified membrane, which was placed on the support made of PET in the above (4), was removed from the PET support, and then placed on the same or different kind of cells cultured beforehand on a petri dish for cell culture made of polystyrene so that the cell surface adhered to the cell culture. The sample was added with the medium and cultured for 24 hours in a CO₂ incubator.

After passage of time, the medium was replaced with the medium for solubilization treatment (Eagle minimum medium added with 1-hydroxyethylidene-1,1-diphosphonic acid at 275 mole % based on the total mol amount of calcium ion and magnesium ion in the Eagle minimum medium) for immersion at 37° C. for 20 minutes in a CO₂ incubator. Then the medium for solubilization treatment was removed. Only the water-containing chitosan gel was removed from the modified membrane of the water-containing chitosan gel/calcium alginate laminated/collagen layer by pinching with tweezers, and the condition the membrane in the delamination from the collagen layer was observed

Subsequently, the sample was added with the medium and cultured at 37° C. for one day in a CO₂ incubator, and then observed under an optical microscope. The results are shown in Table 7. In the Table, the evaluation criteria of delamination property and the cell survival rate were the same as those shown in Example 5.

The water-containing chitosan gel/calcium alginate laminated/collagen layer-modified membrane (the thickness of the alginic acid-coated membrane: 300 μm, the concentration of calcium chloride: 0.02 mol/L) by using S1 or S3 as the chitosan membrane gave a similar result to that of V1 in Table 7, and the water-containing chitosan gel/calcium alginate laminated/collagen layer-modified membrane (the thickness of the alginic acid-coated membrane: 300 μm, the concentration of calcium chloride: 0.02 mol/L) by using S5 as the chitosan membrane gave a similar result to that of V4 in Table 7.

The above results indicate that cell culture using the carrier for cell culture of the present invention successfully achieve satisfactory delamination property and high cell survival rate after the delamination simultaneously.

TABLE 7

| | | | Cell | |
|---|---|---|---|---|
| Sample | Chitosan | Delamination | survival | |
| number | membrane | property | rate | |
| V1 | S2 | ⊚ | ◯ | Present invention |
| V2 | ″ | ◯ | ⊚ | ″ |
| V3 | ″ | ◯ | ⊚ | ″ |
| V4 | S4 | Δ | Δ | Comparative example |
| V5 | ″ | X | ◯ | ″ |
| V6 | S6 | ◯ | ◯ | Present invention |
| V7 | S7 | ◯ | ◯ | ″ |
| V8 | S2 | ⊚ | ⊚ | ″ |
| V9 | ″ | ◯ | ◯ | ″ |
| V10 | ″ | ◯ | ◯ | ″ |

1. A carrier for cell culture having a multi-layer structure comprising a water-containing polymer gel layer, wherein the water-containing polymer gel layer has a thickness of 0.01 μm or more and 5 μm or less.

2. The carrier for cell culture according to claim 1, wherein the water-containing polymer gel in a liquid state has a viscosity of 1,000 mPa•s or more and 50,000 mPa•s or less.

3. The carrier for cell culture according to claim 1, wherein the water-containing polymer gel is chitosan.

4. The carrier for cell culture according to claim 1, which further comprises a gel layer comprising an anionic polysaccharide and a polyvalent metal ion, wherein said gel layer is adjacent to the water-containing polymer gel layer.

5. The carrier for cell culture according to claim 4, wherein the gel layer comprising an anionic polysaccharide and a polyvalent metal ion is an alginic acid gel layer.

6. The carrier for cell culture according to claim 4, wherein the gel layer comprising an anionic polysaccharide and a polyvalent metal ion is calcium alginate gel.

7. The carrier for cell culture according to claim 4, wherein the most outer surface at the side for the cell culture is a cell-adhesive gel layer.

8. The carrier for cell culture according to claim 7, wherein the cell-adhesive gel is a gel comprising gelatin and/or collagen.

9. The carrier for cell culture according to claim 4, wherein a physical reinforcing means is provided on one of the surfaces of the water-containing polymer gel layer.

10. The carrier for cell culture according to claim 7, wherein an part that is unmodified with the cell-adhesive gel layer is provided on the most outer surface at the side for the cell culture.

11. A method for cell culture which comprises the step of culturing cells by using the carrier for cell culture according to claim 1.

12. A method for cell culture which comprises the step of obtaining a cell sheet by a solubilization treatment of the gel layer comprising an anionic polysaccharide and a polyvalent metal ion in the cell culture which is cultured by using the carrier for cell culture according to claim 4.

13. A cell sheet obtainable by the method for cell culture according to claim 12.

14. A cell transfer method which comprises the step of further culturing the cell culture, which is obtainable by the method for cell culture which comprises the step of culturing cells by using the carrier for cell culture according to claim 4, on another carrier for cell culture.

15. A cell culture or a cell sheet obtainable by solubilization treatment of the gel layer comprising an anionic polysaccharide and a polyvalent metal ion in the cell culture obtainable by the cell transfer method according to claim 14.

16. A cell lamination method which comprises the step of culturing the cell culture obtainable by the cell transfer method according to claim 14 on another cell culture.

17. A physical reinforcing means which is provided on one side of one or more of the layers in a carrier for cell culture having multi-layer structure.

18. A medium for solubilization treatment of a gel layer comprising an anionic polysaccharide and a polyvalent metal ion in cell culture by using a carrier for cell culture comprising said layer, which comprises a chelating agent and having a polyvalent metal ion concentration of 2.6 mM or less.

19. A cell lamination method which comprises the step of culturing the cell culture, which is cultured by using the carrier for cell culture according to claim 4, on another cultured cell.

20. A laminated cell culture or a laminated cell sheet obtainable by the solubilization treatment of the gel layer comprising an anionic polysaccharide and a polyvalent metal ion in the cell culture obtainable by the cell lamination method according to claim 19.
